(19) United States
(12) Patent Application Publication
(10) Pub. No.: US 2018/0223245 A1
(43) Pub. Date: Aug. 9, 2018

(54) METHOD FOR THERMAL PERMEABILIZATION OF A MICROALGAE BIOMASS

(71) Applicant: Corbion Biotech, Inc., South San Francisco, CA (US)

(72) Inventors: Damien Passé, Douai (FR); Samuel Patinier, Quesnoy-Sur-Deule (FR); Marilyne Guillemant, Aire Sur La Lys (FR)

(21) Appl. No.: 15/943,665

(22) Filed: Apr. 2, 2018

Related U.S. Application Data
(63) Continuation of application No. 15/126,370, filed on Sep. 15, 2016, now abandoned.

(30) Foreign Application Priority Data
Mar. 18, 2014 (FR) 1452219

(51) Int. Cl. C12N 1/06; C12N 1/12

(52) U.S. Cl. CPC C12N 1/066; C12N 1/12

(57) ABSTRACT

Described are methods for thermal permeabilization of the biomass of microalgae of the Chlorella genus. The methods are useful in recovering the soluble fractions, including polypeptides and saccharide, of the microalgae.

The present invention relates to a process for thermal permeabilization of a microalgal biomass, this treatment making it possible to release therefrom the soluble intracellular content, in particular the peptides and polypeptides.

This thermal permeabilization process is not accompanied by cell disintegration, thereby also allowing easy separation of the intracellular content thus released from the residual biomass, by any solid-liquid separation method known per se moreover to those skilled in the art, for example frontal or tangential filtration, centrifugation and/or flocculation.

More particularly, in the case where the microalgae chosen are rich in lipids, the process of the invention makes it possible to preserve in the residual biomass the lipid fraction of interest.

Finally, the present invention relates to the recovery and fractionation of the intracellular content of the microalgae in aqueous solution, said intracellular content being composed of soluble peptides and polypeptides, pigments, free fatty acids, oligosaccharides and polysaccharides, etc.

It is well known to those skilled in the art that chlorellae are a potential source of food, since they are rich in proteins and other essential nutrients.

They are described as containing 45% of proteins, 20% of fats, 20% of carbohydrates, 5% of fibers and 10% of minerals and vitamins.

The oil fraction of the Chlorella biomass, which is composed essentially of monounsaturated oils, thus provides nutritional and health advantages compared with the saturated, hydrogenated and polyunsaturated oils often found in conventional food products.

The protein fraction can, for its part, be exploited as a functional agent in the food, cosmetic or even pharmaceutical industries, for its foaming, emulsifying, etc., properties.

Chlorellae are thus conventionally utilized in food for human or animal consumption, either in the form of whole biomass or in the form of flour, obtained by drying biomass of chlorellae, the cell wall of which has been broken by in particular mechanical means.

The microalgal flour also provides other benefits, such as micronutrients, dietary fibers (soluble and insoluble carbohydrates), phospholipids, glycoproteins, phytosterols, tocopherols, tocotrienols and selenium.

In order to prepare the biomass which will be incorporated into the food composition, the biomass is concentrated, or harvested, from the culture medium (culturing by photoautotrophy in photobioreactors, or heterotrophically in darkness and in the presence of a source of carbon which can be assimilated by the chlorellae).

In the technical field to which the invention relates, the heterotrophic growth of chlorellae is preferred (what is known as the fermenting route).

At the time of the harvesting of the microalgal biomass from the fermentation medium, the biomass comprises intact cells which are mostly in suspension in an aqueous culture medium.

In order to concentrate the biomass, a solid-liquid separation step is then carried out by frontal and/or tangential filtration, by centrifugation or by any means known, moreover, to those skilled in the art.

After concentration, the microalgal biomass can be treated directly in order to produce vacuum-packed cakes, microalgal flakes, microalgal homogenates, intact microalgal powder, milled microalgal flour or microalgal oil.

The microalgal biomass is also dried in order to facilitate the subsequent treatment or for use of the biomass in its various applications, in particular food applications.

However, up until now, microalgae were used mainly for the production of products with a high added value, but a small volume. Among the reasons put forward to explain this situation are the prohibitive cost of large-scale production of microalgae and especially the difficulties associated with the process for purifying (“DSP” for Down Stream Process) said products.

As stated above, many products with a high added value are stored in the intracellular compartment of microalgae, and the processes for extracting these products conventionally require a cell disintegration step.

However, an efficient cell disintegration process has a duty to maximize not only the yield, but also the quality of the products extracted. In other words, this optimized disintegration process must avoid chemical contamination or degradation of the products targeted.

Moreover, for large-scale productions, it is important for the process chosen to be transposable to this scale.

Finally, the introduction of this cell disintegration step in the DSP must be easy and must not have a negative impact on the subsequent process/treatment steps.

All these limitations influence the efficiency of the disintegration process and by the same token its energy consumption.

Various procedures for disintegration of microalgae have been studied, for example chemical, mechanical, enzymatic or even electrical (pulsed field) procedures.

However, microalgal cells have very solid membrane walls, which makes the cell disintegration and the extraction of the products of interest very difficult and very costly in terms of energy.

For example, a pressure disruptor can be used to pump a suspension containing the cells through a restricted orifice so as to lyze the cells. A high pressure (minimum of 1500 bar) is applied, followed by an instantaneous expansion through a nozzle. The cells can be broken by three different mechanisms: running into the valve, high shear of the liquid in the orifice, and a sudden drop in pressure at the outlet, causing the cell to explode. The method releases intracellular molecules, mixed with the cell debris.

A Niro homogenizer (GEA Niro Soavi or any other high-pressure homogenizer) may be used to treat the cells having a size predominantly between 0.2 and 5 microns. This treatment of the algal biomass under high pressure generally lyzes more than 90% of the cells and reduces the size to less than 5 microns.

Alternatively, a ball mill may also be used. In a ball mill, the cells are agitated in suspension with small spherical particles. The breaking of the cells is caused by the shear forces, the milling between the balls, and the collisions with balls. The description of an appropriate ball mill is, for example, given in the U.S. Pat. No. 5,330,913. These balls break the cells so as to release the cell content therefrom. A suspension of particles of smaller size than the cells of origin is then obtained in the form of an “oil-in-water” emulsion. This emulsion is then atomized and the water is eliminated, leaving a dry powder containing, however, a heterogeneous mixture composed of cell debris, interstitial soluble compounds, and oil.

The difficulty to be solved in the use of these cell disintegration technologies is the isolation of solely the intracellular content (to the exclusion of the membrane debris and the fats) and the preservation, in particular, of the quality of the protein load.

The energy used to break the rigidity of the microalga can in fact bring about an irreversible degradation or denaturation of the intracellular molecules of interest.

Alternative solutions have been proposed, such as pulsed-field electrical treatment. The exposure of biological cells to a high-intensity pulsed electric field can in fact modify the structure of the cell membrane. The external field causes charging of the membrane. At a sufficient transmembrane voltage (0.5-1 V), the molecular arrangement of the phospholipids changes, which results in the membrane losing its barrier role, making it permeable. Depending on the conditions used, this membrane permeabilization can be reversible or irreversible.

For efficient extraction of the intracellular compounds, those skilled in the art remain, however, advised to bring about an irreversible permeabilization of the membrane, thereby resulting in its disintegration.

This rupture of the membrane then facilitates the release of the cell content and, in the case of the use of a supplementary solvent-extraction technique, also facilitates the penetration of the solvent into the cell.

This technique, although promising, can unfortunately not be extrapolated to an industrial scale for treating a biomass produced in a reactor of 1 to 200 m³.

Moreover, it also produces contaminating membrane debris that it will be necessary to separate from the molecules of interest of the intracellular compartment.

As a result, there remains an unmet need to provide a technology for weakening microalgal walls that is capable of releasing the intracellular content without disintegrating the cell or impairing the quality of the components thereof.

The applicant company has found that this need can be met by a process for thermal permeabilization of the microalgal cells.

The applicant company thus goes against a technical prejudice which says that thermal methods for cell disruption, just like the shear forces caused by mechanical disintegration, are technologies that are instead used for degrading or denaturing the products from microalgae (Richmond, 1986, Handbook of Microalgal Mass Culture. CRC Press, Inc—Molina Grima et al., 2003, Recovery of microalgal biomass and metabolites: process options and economics, Biotechnol. Adv. 20:491-515).

Moreover, once released from the intracellular compartment, the recovery of the molecules can be carried out easily by any solid-liquid separation technique known to those skilled in the art, given that the thermal treatment developed by the applicant company does not result in the disintegration of the cell wall.

Finally, the recovery of this soluble fraction opens the way to fractionation of its content, for example by membrane separation techniques known to those skilled in the art.

The present invention relates to a process for thermal permeabilization of the biomass of microalgae of the Chlorella genus in such a way as to recover therefrom the soluble fractions which are enriched in particular with peptides and polypeptides and with oligosaccharides.

This process comprises the following steps:
provision of a microalgal biomass;
heat treatment in steps at a temperature of between 60 and 130° C., preferably 60 and 90° C., for 1 to 5 minutes,
cooling to a temperature of between 0° and 10° C., and
recovery, concentration and enrichment of the soluble fractions from which the microalgal cells have been removed.

This process preferably comprises the following steps:
1) culturing of the microalgae by fermentation under heterotrophic conditions and in the absence of light,
2) collection of the biomass,
3) optionally, washing of the biomass in order to remove the residues of the interstitial fluid that result from the fermentation,
4) heat treatment in steps at a temperature of between 60 and 130° C., preferably 60 and 90° C., for 1 to 5 minutes,
5) optionally, cooling to ambient temperature and maintaining at this temperature for 30 minutes to 3 hours, so as to allow the intracellular components to diffuse into the reaction medium,
6) cooling to a temperature between 0° and 10° C., preferably to a temperature of about 4° C.,
7) elimination of the residual biomass by means of a solid-liquid separation technique,
8) recovery, concentration and enrichment of the soluble fractions from which the microalgal cells have been removed.

Preferably, the microalgae of the Chlorella genus are chosen from the group consisting of Chlorella vulgaris, Chlorella sorokiniana and Chlorella protothecoides, and are more particularly Chlorella protothecoides. In one particular embodiment, the strain is Chlorella protothecoides (strain UTEX 250—The Culture Collection of Algae at the University of Texas at Austin—USA). In another particular embodiment, the strain is Chlorella sorokiniana (strain UTEX 1663—The Culture Collection of Algae at the University of Texas at Austin—USA).

The culturing under heterotrophic conditions and in the absence of light conventionally results in the production of a chlorella biomass having a protein content (evaluated by measuring the nitrogen content N×6.25) of 45% to 70% by weight of dry cells.

As will be exemplified hereinafter, this culturing is carried out in two steps:
preculture in a medium containing glucose and yeast extract for 72 h at 28° C. with shaking,
culture for production of the biomass per se in glucose and yeast extract for more than 36 h at 28° C., with shaking and at pH 6.5 adjusted with aqueous ammonia, which results in approximately 80 g/l of biomass with a protein content (evaluated by N 6.25) of about 52% by weight of dry cells.

The biomass is then collected by solid-liquid separation, by frontal or tangential filtration, by centrifugation or by any means known, moreover, to those skilled in the art.

Advantageously, the applicant company then recommends washing the biomass in such a way as to eliminate the interstitial soluble compounds by a succession of concentration (by centrifugation)/dilution of the biomass.

For the purposes of the invention, the term “interstitial soluble compounds” is intended to mean all the soluble organic contaminants of the fermentation medium, for example the water-soluble compounds such as the salts, the residual glucose, the oligosaccharides with a degree of polymerization (or DP) of 2 or 3, the peptides, etc.

This biomass thus purified of its interstitial soluble substances is then preferentially adjusted to a dry matter of between 5% and 35% by weight, preferably to a dry matter of between 10% and 20% with demineralized water.

The heat treatment in steps at a temperature of between 60° and 130° C., preferably 60 and 90° C., for 1 to 5 minutes, is then carried out. This treatment can comprise 2 to 6 temperature steps. For example, it can comprise several steps of increasing temperatures and then, optionally, several steps of decreasing temperatures. The temperature steps may be from 10 to 40° C., for example approximately 10, 20, 30 or 40° C. A first step can make it possible to bring the biomass to a temperature of approximately 60-70° C. The term “approximately” is intended to mean + or −10%, preferably + or −5%. Intermediate steps can be carried out between 60° C. and the maximum temperature applied, for example between approximately 90 and 130° C. Each step can last between approximately 10 seconds and 4 minutes, preferably between 30 seconds and 3 minutes.

Thus, the treatment can comprise a first step making it possible to bring the biomass to a temperature of approximately 60-70° C., one or more steps making it possible to reach a maximum temperature applied of approximately 90 to 130° C., and optionally one or more steps making it possible to reduce the temperature.

As will be exemplified hereinafter, the treatment can be carried out in three phases:
increase in temperature from ambient temperature to 60° C. in 30 seconds;
increase in temperature from 60° to 90° C. for a further 30 seconds;
maintenance of the temperature at 90° C. for 3 minutes.

In one particular embodiment, the process comprises the following steps:
raising of the temperature from 28° C. to 60° C. for 30 seconds,
raising of the temperature from 60° C. to 90° C. for 30 seconds,
maintenance of the temperature at 90° C. for 1 minute,
cooling from 90 to 60° C. for 30 seconds,
cooling from 60° C. to 4° C., preferably for 1 minute.

This treatment makes it possible to allow the intracellular components to diffuse into the reaction medium.

It is possible to allow the temperature to cool to ambient temperature and to be maintained at this temperature for 30 minutes to 3 hours in such a way as to amplify this free diffusion phenomenon.

Finally, at the end of these steps, the temperature is allowed to cool to a final temperature of between 0° and 10° C., preferably to a temperature of about 4° C.

The applicant company has thus found that the heat treatment, carried out under these operating conditions, thus acts as a membrane weakening process which allows the spontaneous release of the soluble components of the intracellular compartment.

In addition to the ionic substances, organic substances such as carbohydrates (predominantly DP1 and DP2), the peptides and the polypeptides are drained out of the cell.

Conversely, the lipids and hydrophobic organic compounds remain in the cells, thereby clearly demonstrating that the cells are permeabilized and not lyzed/destroyed.

The process according to the invention does not therefore result in the formation of an emulsion, but indeed of an aqueous suspension.

The release of all these soluble substances through the permeabilized membrane is similar to a process of free diffusion of dialysis type.

Consequently, a lag time may be necessary in order to allow sufficient diffusion after the heat treatment which permeabilizes the membrane.

In the literature, the process for pulsed-field permeabilization of yeast membranes in order to extract therefrom the proteins requires from 4 h to 6 h (Ganeva et al., 2003, Analytical Biochemistry, 315, 77-84).

According to the invention, a much shorter reaction time is used, of between 1 and 5 minutes.

Advantageously, a further reaction time of between 30 minutes and 3 hours may be used in order to optimize the diffusion of the soluble compounds of the cell compartment.

The residual biomass is then eliminated by a technique of solid-liquid separation by frontal or tangential filtration, by flocculation, by centrifugation or by any means known, moreover, to those skilled in the art, thereby making it possible to easily recover the soluble fraction from which the microalgal cells have been removed.

This soluble fraction is essentially composed of proteins (50-80% w/w) and carbohydrates (5-15% w/w).

The conventional processes for recovering soluble proteins are generally based on a step of precipitating said proteins with trichloroacetic acid (10% weight/volume) or with ammonium sulfate.

However, these isolations by precipitation follow on from very destructive cell-breaking processes (usually by sonication or homogenization) which, while they make it possible in fact to increase extraction yields, result especially in proteins of low solubility which are denatured.

It is then possible to envision the refunctionalization thereof only by means of their product of hydrolysis (to peptides) by chemical means (lysis with sodium hydroxide), physical means (high-temperature treatment) or enzymatic means (proteolytic enzymes).

The process according to the invention makes it possible, quite the contrary, to release intact native peptides and polypeptides, all the functionalities of which can still be expressed.

Moreover, the applicant company has found that the size of the soluble peptides and polypeptides released varies proportionally to the treatment temperature used. It is also considered that the treatment time may have an impact.

Fractionation processes are moreover proposed by the applicant company in order to isolate the proteins or the oligosaccharides of interest, said fractionation processes being mainly membrane fractionation processes.

The applicant company thus recommends carrying out the process in two steps:
preparing compositions which are enriched with soluble proteins and with oligosaccharides from the soluble fractions (from which the heat-treated microalgae have been removed) filtered on a membrane system chosen from the group consisting of microfiltration, ultrafiltration, nanofiltration and diafiltration, taken alone or in combination,
subjecting said compositions to additional membrane fractionation treatments of reverse osmosis type in order to separate, on the one hand, peptides and polypeptides and, on the other hand, oligosaccharides.

The invention will be understood more clearly from the following examples which are intended to be illustrative and nonlimiting.

### EXAMPLES

### Example 1: Production of Chlorella protothecoides by Fed-Batch Fermentation

The strain used is Chlorella protothecoides UTEX 250

Preculture:
500 ml of medium in a 2 l Erlenmeyer flask;
Composition of the medium (in g/l):

TABLE 1

| | | |
|---|---|---|
| Macro | Glucose | 40 |
| elements (g/l) | K₂HPO₄ | 3 |
| | Na₂HPO₄ | 3 |
| | MgSO₄•7H2O | 0.25 |
| | (NH₄)₂SO₄ | 1 |
| | Citric acid | 1 |
| | clerol FBA 3107 (antifoam) | 0.1 |
| Microelements | CaCl₂•2H2O | 30 |
| and Vitamins | FeSO4•7H2O | 1 |
| (mg/l) | MnSO4•1H2O | 8 |
| | CoSO4•7H2O | 0.1 |
| | CuSO4•5H2O | 0.2 |
| | ZnSO4•7H2O | 0.5 |
| | H3BO3 | 0.1 |
| | Na2MoO4•2H2O | 0.4 |
| | Thiamine HCl | 1 |
| | Biotin | 0.015 |
| | B12 | 0.01 |
| | Calcium pantothenate | 0.03 |
| | p-aminobenzoic acid | 0.06 |

Incubation is carried out under the following conditions: duration: 72 h; temperature: 28° C.; shaking: 110 rpm (Infors Multitron incubator).

The preculture is then transferred to a 30 l Sartorius type fermenter.

Culture for Biomass Production:

The medium is as follows:

TABLE 2

| | | |
|---|---|---|
| Macro | Glucose | 40 |
| elements (g/l) | K₂HPO₄ | 1.8 |
| | NaH₂PO₄ | 1.4 |
| | MgSO₄•7H2O | 3.4 |
| | (NH₄)₂SO₄ | 0.2 |
| | clerol FBA 3107 (antifoam) | 0.3 |
| Microelements | CaCl₂•2H2O | 40 |
| and Vitamins | FeSO4•7H2O | 12 |
| (mg/l) | MnSO4•1H2O | 40 |
| | CoSO4•7H2O | 0.1 |
| | CuSO4•5H2O | 0.5 |
| | ZnSO4•7H2O | 50 |
| | H3BO3 | 15 |
| | Na2MoO4•2H2O | 2 |
| | Thiamine HCl | 6 |
| | Biotin | 0.1 |
| | B12 | 0.06 |
| | Calcium pantothenate | 0.2 |
| | p-aminobenzoic acid | 0.2 |

The initial volume (Vi) of the fermenter is adjusted to 17 l after inoculation. It is brought to a final volume of approximately 20-25 l.

The parameters for carrying out the fermentation are as follows:

TABLE 3

| | |
|---|---|
| Temperature | 28° C. |
| pH | 5.0-5.2 by 28% w/w NH₃ |
| pO₂ | 20% +/− 5% (maintained by shaking) |
| Shaking | Minimum 300 rpm |
| Air flow rate | 15 l/min |

When the residual glucose concentration falls below 10 g/l, glucose in the form of a concentrated solution at approximately 800 g/l is introduced so as to maintain the glucose content between 0 and 20 g/l in the fermenter.

Results

In 40 h, 80 g/l of biomass containing 52% of proteins are obtained.

### Example 2. Thermal Permeabilization of the Chlorella protothecoides Biomass and Recovery of the Soluble Fraction

The biomass obtained according to example 1 is:
centrifuged and washed so as to be brought to a dry matter content of 150 g/l and to a purity of more than 90% (purity defined by the ratio of the dry matter of the biomass to the total dry matter), then
heat treated in the following way:
   raising of the temperature from 28° C. to 60° C. for 30 seconds,
   raising of the temperature from 60° C. to 90° C. for 30 seconds,
   maintenance of the temperature at 90° C. for 1 minute,
   cooling from 90 to 60° C. for 30 seconds,
   cooling from 60° C. to 4° C., preferably for 1 minute.

The biomass thus obtained is separated from the soluble fraction by centrifugal separation. Said soluble fraction is then microfiltered on a 0.14 μm ceramic membrane at 60° C.

The transmembrane pressure is fixed at a value of between 0.2 and 0.6 bar and the microfiltration is carried out so as to obtain a volume concentration factor of 2.5 (100 l of this microfiltered soluble fraction thus generate 40 liters of retentate “R1” and 60 liters of permeate “P1”).

This microfiltration permeate “P1” has a dry matter content of 4% and a titer between 60% and 80% of soluble proteins, expressed as N×6.25.

### Example 3. Fractionation of the Protein and Saccharide Components of the Intracellular Content of the Permeabilized Chlorella protothecoides Biomass

In order to obtain the fractions which are rich in soluble proteins and in saccharides, the microfiltration permeate “P1” obtained at the end of example 2 having a dry matter content of 4% is in particular ultrafiltered on a membrane with a cut-off threshold of 10 kDa, so as to obtain:
a retentate “R2” having a dry matter content of 10%, containing peptides having a molecular weight greater than or equal to 5 kDa and oligosaccharides having high DPs;
a permeate “P2” having a dry matter content of 1%, containing peptides having a molecular weight less than 5 kDa and oligosaccharides having a DP less than or equal to 2.

This permeate “P2” can then be in particular filtered on a reverse osmosis membrane (having a degree of NaCl rejection of 93%), so as to obtain:
a retentate “R3” having a dry matter content of 10%, containing peptides having a molecular weight less than 5 kDa and oligosaccharides of DP 2, such as sucrose;
a permeate “R3” having a dry matter content of 0.1%, containing oligosaccharides of DP 1, salts, free amino acids and organic acids.

1.-10. (canceled)

11. A process for thermal permeabilization of biomass of microalgae of the Chlorella genus, which comprises the following steps:
Provision of a microalal biomass;
heat treatment in steps at a temperature of between 60 and 130° C., for 1 to 5 minutes,
cooling to a temperature of between 0° and 10° C., and
recovery, concentration and enrichment of the soluble fractions from which the microalgal cells have been removed,
thereby recovering from said microalgae the soluble fractions which are enriched in particular with proteins and with oligosaccharides.

12. The thermal permeabilization process of claim 11, which comprises the following steps:
(1) culturing of the microalgae by fermentation under heterotrophic conditions and in the absence of light,
(2) collection of the biomass,
(3) washing of the biomass in order to remove the residues of the interstitial fluid that result from the fermentation,
(4) heat treatment in steps at a temperature of between 60° and 130° C., for 1 to 5 minutes,
(5) cooling to ambient temperature and maintaining at this temperature for 30 minutes to 3 hours, so as to allow the intracellular components to diffuse into the reaction medium,
(6) cooling to a temperature between 0° C. and 10° C.,
(7) elimination of the residual biomass by means of a solid-liquid separation technique,
(8) recovery, concentration and enrichment of the soluble fractions from which the microalgal cells have been removed.

13. The process of claim 11, wherein the microalgae of the Chlorella genus are chosen from the group consisting of Chlorella vulgaris, Chlorella sorokiniana and Chlorella protothecoides.

14. The process of claim 11, wherein the biomass is collected by solid-liquid separation, by frontal or tangential filtration or by centrifugation.

15. The process of claim 11, wherein the heat treatment comprises several steps of increasing temperatures.

16. The process of claim 11, wherein the heat treatment comprises a first step making it possible to bring the biomass to a temperature of approximately 60-70° C., one or more steps making it possible to reach a maximum temperature applied of approximately 90 to 130° C.

17. The process of claim 11, wherein the heat treatment comprises the following phases:
increase in temperature from ambient temperature to 60° C. for 30 seconds,
increase in temperature from 60° C. to 90° C. for a further 30 seconds,
maintenance of the temperature at 90° C. for 3 minutes.

18. The process of claim 11, wherein the heat treatment comprises the following phases:
raising of the temperature from 28° C. to 60° C. for 30 seconds,
raising of the temperature from 60° C. to 90° C. for 30 seconds,
maintenance of the temperature at 90° C. for 1 minute,
cooling from 90 to 60° C. for 30 seconds,
cooling form 60° C. to 4° C.

19. A process for preparing compositions which are enriched with soluble peptides and polypeptides and with oligosaccharides from microalgae of the Chlorella genus, wherein the soluble fractions obtained by carrying out the process of claim 11 are filtered on a membrane separation system chosen from the group consisting of microfiltration, ultrafiltration, nanofiltration and diafiltration, taken alone or in combination.

20. A process for fractionating the compositions which are enriched with soluble peptides and polypeptides and with oligosaccharides obtained as claimed in claim 19, wherein membranes of reverse osmosis type are used.

21. The process of claim 11, wherein the temperature of the heat treatment step is between 60° and 90° C.

22. The process of claim 12, wherein the heat treatment temperature is between 60° and 90° C., and the cooling step is performed at a temperature of about 4° C.

23. The process of claim 15, wherein the heat treatment further comprises several steps of decreasing temperatures, each step being from 10 to 40° C.

24. The process of claim 16, wherein the heat treatment further comprises one or more steps making it possible to reduce the temperature.

25. The process of claim 18, wherein the cooling from 60° C. to 4° C. is performed for 1 minute.
